(19) United States
(12) Patent Application Publication
(10) Pub. No.: US 2015/0150994 A1
(43) Pub. Date: Jun. 4, 2015

(54) LIVER TARGETED DRUG DELIVERY SYSTEMS USING METAL NANOPARTICLES AND PREPARING METHOD THEREOF

(71) Applicant: POSTECH ACADEMY-INDUSTRY FOUNDATION, Pohang-si (KR)

(72) Inventors: Sei Kwang Hahn, Pohang-si (KR); Min Young Lee, Pohang-si (KR); Jeonga Yang, Pohang-si (KR); Ho Sang Jung, Phoang-si (KR)

(21) Appl. No.: 14/401,922

(22) PCT Filed: May 21, 2013
(86) PCT No.: PCT/KR2013/004454

§ 371 Date: Nov. 18, 2014

(30) Foreign Application Priority Data
May 23, 2012 (KR) 10-2012-0054787
Apr. 29, 2013 (KR) 10-2013-0047574

(51) Int. Cl. A61K 47/48; A61K 38/21

(52) U.S. Cl. CPC A61K 47/48015; A61K 47/4823; A61K 38/212

(57) ABSTRACT

The present invention relates to liver targeted drug delivery system using metal nanoparticles and a method for preparing the same. More particularly, the present invention relates to a method for preparing hyaluronic acid-gold nanoparticles/protein complex that can be used as liver targeted drug delivery system, by surface modifying gold nanoparticles having excellent stability in the body with hyaluronic acid having biocompatibility, biodegradability and liver tissue-specific delivery property, and binding protein drugs for treating liver diseases to the non-modified surface of the gold nanoparticles. And, the present invention relates to use of the hyaluronic acid-gold nanoparticles/protein complex for liver disease drug that may be safely applied to human body, increase drug efficacy duration time, and be effectively delivered to liver.

### TECHNICAL FIELD

The present invention relates to liver targeted drug delivery system using metal nanoparticles and a method for preparing the same. More particularly, the present invention relates to a method for preparing hyaluronic acid-gold nanoparticles/protein complex that can be used as liver targeted drug delivery system, by surface modifying gold nanoparticles having excellent stability in the body with hyaluronic acid having biocompatibility, biodegradability and liver tissue-specific delivery property, and binding protein drugs for treating liver diseases to the non-modified surface of the gold nanoparticles. And, the present invention relates to use of the hyaluronic acid-gold nanoparticles/protein complex for liver disease drug that may be safely applied to human body, increase drug efficacy duration time, and be effectively delivered to liver.

### BACKGROUND ART

Until recently, the development of protein drugs has been focused on the development of conjugate dosage form covalently bonded to polymer having biocompatibility or biodegradability, to maintain long term drug efficacy and increase duration time. The drug efficacy duration time of protein drugs may be extended to several weeks according to the type of dosage form and conjugated active ingredient.

Among them, studies on the application of polyethylene glycol (PEG) or hyaluronic acid (HA) having excellent biocompatibility and biodegradability for drug delivery system by covalent bonding to protein drugs are being actively progressed.

However, it has been reported that if polyethyleneglycol-liposome (PEG-Liposome) conjugate used as drug delivery system is repeatedly injected, ‘accelerated blood clearance’ wherein administered drug is rapidly eliminated in the body may occur. And, although the pegylated dosage form of interferon alpha, which is protein drug for treating liver disease, is commercialized as a weekly injection dosage form, when it is repeatedly injected to treat hepatitis C, many patients may discontinue it during treatment due to serious side effects, and just 50% antiviral effect may be exhibited in the case of genotype 1.

Meanwhile, if liver tissue-specifically delivered hyaluronic acid is covalently bonded to an active ingredient to prepare drug delivery system for treating liver disease, there has been a limitation due to low bioconjugation efficiency.

And, it has been reported that in the case wherein protein drug is covalently bonded to polymer, polymer may non-specifically react with various reaction groups of the various amino acid sequences of the protein to react on the functional groups important for bioactivity, or break a tertiary structure of protein to lower bioactivity, and as the molecular weight of the polymer increases, bioactivity decreases.

Meanwhile, gold nanoparticles are known to have excellent biocompatibility, and various studies for binding biomolecules thereto are being progressed because the particle size may be controlled and surface modification may be easily achieved.

### DISCLOSURE

### Technical Problem

It is an object of the present invention to provide liver targeted drug delivery system and a method for preparing the same, which may be targetedly delivered to liver while maximally maintaining bioactivity of protein drug, using binding property of protein drug to the surface of gold nanoparticles and liver tissue specific delivery property of hyaluronic acid.

More specifically, it is an object of the present invention to provide liver targeted drug delivery system comprising metal nanoparticles surface modified by dextran, heparin, hyaluronic acid, a salt thereof, or a derivative thereof, and peptide or protein drug bound to the non-modified surface of the metal nanoparticle.

It is another object of the present invention to provide a method for preparing the liver targeted drug delivery system.

### Technical Solution

As the results of studies for providing effective drug delivery system enabling liver targeted delivery while maintaining stability of protein drug, the inventors confirmed that by modifying the surface of gold nanoparticles having excellent stability in the body with dextran, heparin or hyaluronic acid having biocompatibility, biodegradability and liver tissue specific delivery property, and binding protein drugs for treating liver diseases to the non-modified surface of the gold nanoparticles, the drug may be safely applied to human body, and the dextran, heparin or hyaluronic acid may remarkably decrease decomposition of metal nanoparticles-bound protein drug by protease, and thus, protein drug may be effectively delivered to liver while maximally maintaining bioactivity, thereby providing liver targeted drug delivery system that may further increase drug delivery efficiency and drug efficacy duration time, and completed the invention.

Hereinafter, the present invention will be explained in detail.

According to one embodiment of the invention, there is provided liver targeted drug delivery system comprising metal nanoparticles surface modified by dextran, heparin, hyaluronic acid, a salt thereof, or a derivative thereof, and peptide or protein drug bound to the non-modified surface of the metal nanoparticle.

According to another embodiment of the invention, there is provided a method for preparing liver targeted drug delivery system comprising

(1) introducing material having an end amine group and an internal disulfide bond or catecholamine based material in dextran, heparin, hyaluronic acid, a salt thereof, or a derivative thereof;

(2) reacting the introduced material with the surface of metal nanoparticles to prepare surface-modified metal nanoparticles; and

(3) binding peptide or protein drug to the non-modified surface of the metal nanoparticles.

Preferably, the step (1) may include introducing material having an end amine group and an internal disulfide bond or catecholamine based material in hyaluronic acid, a salt thereof, or a derivative thereof to prepare hyaluronic acid derivative of the following Chemical Formula 1.

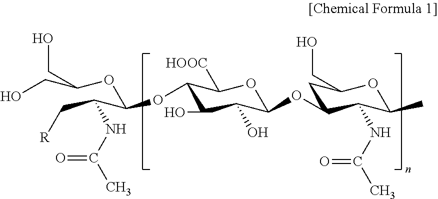

in the Chemical Formula 1,

n is an integer of from 12 to 50, R is NH(CH₂)mR², m is an integer of from 2 to 10, and R² is C₆H₁₂O₂ (catechol) or SH (thiol).

Hyaluronic acid, heparin, or dextran existing in most animals is linear polysaccharide polymer without biodegradability, biocompatibility, and immune response, and it may be variously used since it performs various roles in the body according to the molecular weight. For example, it may control degradation time in the body because it is easily surface modified by a chemical method, and it may be used to control reaction degree of cells with a receptor. According to the liver targeted drug delivery system of the present invention, decomposition of protein drug may be effectively reduced when delivered in the body, thus remarkably improving drug stability and delivery efficiency.

The dextran, heparin, hyaluronic acid, a salt thereof, or a derivative thereof may preferably have molecular weight of 5,000 to 20,000 Da, but is not limited thereto. The hyaluronic acid, a salt thereof, or a derivative thereof having molecular weight of the above range is suitable for surface modification of metal nanoparticles.

As used herein, the term ‘hyaluronic acid (HA)’ refers to linear polymer polysaccharide including dissacharide repeat unit wherein β-D-N-acetylglucosamine and β-D-glucuronic acid are alternatively bonded by β-1,3 and β-1,4 bonds, and hyaluronic acid salt includes various salt forms of hyaluronic acid, and for example, it may be an inorganic salt such as cobalt hyaluronate, magnesium hyaluronate, zinc hyaluronate, calcium hyaluronate, potassium hyaluronate, sodium hyaluronate, and the like, and an organic salt such as tetrabutylammonium hyaluronate, and the like. And, hyaluronic acid derivative refers to polymer wherein at least one carboxylic acid group of hyaluronic acid is substituted by other compounds.

As used herein, the term ‘dextran’ refers to a kind of D-glucose-polymerized polysaccharide, and dextran salt includes various salt forms of dextran, for example, it may be dextran sulfate, dextran sulfate sodium salt, and the like. And, dextran derivative refers to polymer wherein at least one hydroxyl group of dextran is substituted by other compounds.

As used herein, the term ‘heparin’ refers to a kind of acidic polysaccharide having a sulfuric acid group wherein D-glucosamine and D-glucuronic acid alternatively form a chain by a-1,4 bond, and heparin salt includes various salt forms of heparin, for example, it may be heparin calcium, heparin lithium, heparin potassium, and the like. And, heparin derivative refers to polymer wherein at least one carboxylic acid group of heparin is substituted by other compounds.

Unless otherwise described, the terms dextran, heparin, hyaluronic acid, and the like are used to include salts thereof or derivatives thereof.

The metal nanoparticle may be preferably gold nanoparticle, silver nanoparticle, or magnetic nanoparticle. And, preferably, the metal nanoparticle may have particle size of 10 nm or more, more preferably 10 nm to 30 nm.

The magnetic nanoparticles may include iron oxide (for example, Fe₂O₃, Fe₃O₄, and the like), ferrite (for example, CoFe₂O₄, MnFe₂O₄, and the like), and alloys (for example, alloys with noble metal such as FePt, CoPt, and the like, considering oxidation problem caused by magnetic atoms, and for increasing conductivity and stability), but are not limited thereto.

The dextran, heparin, hyaluronic acid, a salt thereof, a derivative thereof introduced in the metal nanoparticles may be preferably bound in a molecular number of 10 to 100 per one metal nanoparticle, thereby modifying the surface of the metal nanoparticles.

Preferably, a hyaluronic acid derivative having the following Chemical Formula 1 may be bound to the surface of the metal nanoparticles through an end functional group represented by R in the Chemical Formula 1 so that the metal nanoparticles may be surface modified.

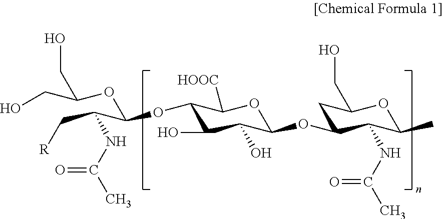

In the Chemical Formula 1, n is an integer of from 12 to 50, R is NH(CH₂)mR², m is an integer of from 2 to 10, and R² is C₆H₁₂O₂ (catechol), or SH.

The hyaluronic acid derivative having the Chemical Formula 1 may be preferably prepared by introducing material having an end amine group and an internal disulfide bond or catecholamine based material at the end of hyaluronic acid, hyluronic acid salt, or hyaluronic acid derivative.

As the material having an end amine group and an internal disulfide bond, at least one selected from the group consisting of 2,2′-disulfanediyldiethanamine, cystamine, 3,3′-disulfanediyldipropan-1-amine, 4,4′-disulfanediyldibutan-1-amine, 5,5′-disulfanediyldipentan-1-amine may be used, and as the catecholamine based material, at least one selected from the group consisting of dopamine, norepinephrine, and a salt thereof may be preferably used.

And, the material may be introduced at the end of dextran, heparin, hyaluronic acid, a salt thereof, or a derivative thereof through reductive amination using sodium cyanoborohydride (NaCNBH₃).

The introduction step may be preferably conducted in a reaction solvent such as sodium borate buffer of pH 8.5˜9.5, PBS buffer or Tris-HCl buffer, and preferably, it may be conducted by reacting at 37˜40° C. for 3 to 5 days. Wherein 1 to 2 moles of the material having an end amine group and an internal disulfide bond may be preferably used per unit of dextran, heparin, hyaluronic acid, a salt thereof, or a derivative thereof. And, 0.1 to 0.2 moles of the catecholamine based material may be preferably used per unit of dextran, heparin, hyaluronic acid, a salt thereof, or a derivative thereof.

If material having an end amine group and an internal disulfide bond is used as introduced material, the step (1) may preferably include (1-1) introducing material having an end amine group and internal disulfide bond at the end of dextran, heparin, hyaluronic acid, a salt thereof, or a derivative thereof; and (1-2) cutting the disulfide bond formed through the step (1-1) using at least one reducing agent selected from the group consisting of dithiothreitol (DTT), 2-mercaptoethanol, and tris(2-carboxyethyl) phosphine, (TCEP).

As such, in the step (1-2), a disulfide bond formed when material having an amine group an internal disulfide bond is used as introduced material is cut, thereby introducing a functional group that can be bonded to the surface of metal nanoparticles such as free thiol group at the end of dextran, heparin, hyaluronic acid, a salt thereof, or a derivative thereof, preferably at the end of hyaluronic acid of the Chemical Formula 1.

Thus, the reducing agent used in the step (1-2) is not specifically limited as long as it may cut a disulfide bond, but preferably, it may include at least one selected from the group consisting of dithiothreitol (DTT), 2-mercaptoethanol, and tris(2-carboxyethyl) phosphine (TCEP). And, the step (1-2) may be preferably conducted at 20 to 30° C., more preferably at 25° C. for 12 to 24 hours.

Meanwhile, if catecholamine based material such as dopamine or norepinephrine, and the like is used as the introduced material, catechol is introduced at the end of dextran, heparin, hyaluronic acid, a salt thereof, or a derivative thereof, and thus, it may be directly bonded to gold nanoparticles without conducting a subsequent step such as (1-2).

The prepared polysaccharide derivative, preferably the end of hyaluronic acid derivative of the Chemical Formula 1 (R in the Chemical Formula 1) may be reacted with metal nanoparticles, preferably in a molecular number of 10 to 100 per one metal nanoparticle, to prepare surface modified metal nanoparticles (step (2)).

The hyaluronic acid derivative of the Chemical Formula 1 may be bonded to the metal nanoparticles in a molecular number of 10 to 100 per one metal nanoparticle, thereby modifying the surface of the metal nanoparticles.

As such, by using metal nanoparticles surface modified by dextran, heparin or hyaluronic acid, and the like having liver tissue specific delivery property, liver targeted drug delivery system that may be safely applied to the human body, increase drug efficacy duration time, and be effectively delivered to liver while maximally maintaining bioactivity of protein drug may be provided, and it may be variously applied for development of liver disease drugs.

The peptide or protein drug delivered through the liver targeted drug delivery system according to the present invention may be provided in the form of being bonded through the interaction of the non-modified surface of the metal nanoparticles and amino acid constituting the drug.

Preferably, the peptide or protein drug may be bonded in a molecular number of 10 to 200 per one metal nanoparticles, wherein the bond may be preferably covalent or non-covalent bond, and the non-covalent bond may be preferably physical bonding such as electrostatic bonding and/or hydrophobic bonding.

Thus, various peptide or protein drugs may be used without specific limitations as long as it may be covalently or non-covalently bonded to the surface of the metal nanoparticles, but preferably, the peptide or protein drug may be covalently bonded to the surface of the metal nanoparticles, and it may include cysteine, particularly cysteine having a free thiol group that does not form a disulfide bond in the amino acid constituting the drug.

Meanwhile, if the peptide or protein drug does not include the above explained cysteine, or if the peptide or protein drug includes cysteine in the amino acid constituting the drug but is disulfide-bonded without a free thiol group, it may be preferably provided in the form of being non-covalently bonded to the surface of the metal nanoparticles, preferably physically bonded such as electrostatically and/or hydrophobically bonded. In this case, the peptide or protein drug may preferably include at least one amino acid selected from the group consisting of tyrosine, lysine, aspartic acid, arginine, hystidine, and tryptophan in the amino acid constituting the drug.

Preferably, the peptide or protein drug may be a drug for preventing or treating liver diseases such as acute hepatitis, chronic hepatitis, liver cirrhosis, cirrhosis, fatty liver, or liver cancer, and the like. More preferably, protein for treating liver disease (hepatitis C) such as interferon alpha, TNF-related apoptosis-inducing ligand, vascular adhesion protein 1, hepatocyte growth factor, and the like may be provided while being bonded to the surface of the metal nanoparticles by covalent or non-covalent bonding, preferably physical bonding such as electrostatic bonding and/or hydrophobic bonding, and the like. Since the drug delivery system according to one preferred embodiment of the invention may achieve very efficient liver targeted drug delivery even if protein drug is bonded through physical bonding instead of covalent bonding (see <Experimental Example 9>), it may be widely applied to various kinds of protein drugs.

In the method for preparing liver targeted drug delivery system according to one preferred embodiment of the invention, in the step (3), peptide or protein drug is covalently bonded to the non-modified surface of the metal nanoparticles, wherein the peptide or protein drug may include cysteine that does not form a disulfide bond in the amino acid constituting the same.

Preferably, in the step (3), peptide or protein drug is non-covalently bonded, preferably electristatically and/or hydrophobically bonded to the non-modified surface of the metal nanoparticles, wherein the peptide or protein drug may include at least one amino acid selected from the group consisting of tyrosine, lysine, aspartic acid, arginine, hystidine, and tryptophan in the amino acid constituting the same.

As explained above, the liver targeted drug delivery system according to one preferred embodiment of the invention may be safely applied to the human body, increase drug efficacy duration time, and be effectively delivered to liver while maximally maintaining bioactivity of protein drug, by surface modifying gold nanoparticles having excellent stability in the body with hyaluronic acid having biocompatibility, biodegradability and liver tissue specific delivery property, and binding various protein drugs for treating liver diseases to the non-modified surface of the metal nanoparticles. Thus, according to another embodiment of the invention, a pharmaceutical composition for preventing or treating liver disease comprising the liver targeted drug delivery system is provided.

The pharmaceutical composition may further include pharmaceutically acceptable carrier in addition to the liver targeted drug delivery system, and besides, additives, excipient, stabilizer, and the like may be appropriately selected by one of ordinary knowledge in the art, wherein the liver disease is not specifically limited but preferably it may be acute hepatitis, chronic hepatitis, liver cirrhosis, cirrhosis, fatty liver, or liver cancer.

### Advantageous Effects

The liver targeted drug delivery system and a method for preparing the same according to the present invention may be applied to various protein drugs that can be bonded to metal nanoparticles, and it may be variously applied as more effective and safer liver disease drug using metal nanoparticles such as gold nanoparticles having excellent biocompatibility, and hyaluronic acid having biocompatibility, biodegradability and liver tissue specific delivery property. And, the activity of the protein drug bonded to the liver targeted drug delivery system according to one embodiment of the invention is less decreased compared to the activity of the original protein drug, and protein drug may be released from the metal nanoparticles in the body over time, thus further increasing the activity of protein. The liver targeted drug delivery system of the present invention is expected to be applied as a treating agent for liver disease such as hepatitis and liver cancer, and the like.

### DESCRIPTION OF DRAWINGS

FIG. 1 schematically shows a chemical scheme for the preparation method of HA-AuNP/IFNa complex according to Example 1.

FIG. 2a shows the analysis result of AuNP/IFNa complex using UV-Vis absorbance spectra according to Experimental Example 1.

FIG. 2b shows the analysis result of HA-AuNP/IFNa complex using UV-Vis absorbance spectra according to Experimental Example 1.

FIG. 3a shows the analysis results of AuNP, AuNP/IFNa, HA-AuNP and HA-AuNP/IFNa complexes using DLS according to Experimental Example 1.

FIG. 3b shows analysis results of AuNP/IFNa and HA-AuNP/IFNa complexes through TEM image according to Experimental Example 1.

FIG. 4 shows the result of quantifying the amount of IFNa forming the complex as the ratio of IFNa to HA-AuNP increases, using fluorescence analysis and ELISA according to Experimental Example 2.

FIG. 5 shows the result of analyzing the amount of released IFNa, when HA-AuNP/IFNa is treated with Tween 20 and MgCl2 according to Experimental Example 3.

FIG. 6a shows the Circular Dichroism results of IFNa and HA-AuNP/IFNa according to Experimental Example 4, and FIG. 6b shows the CD result of released IFNa after treating with Tween 20 and MgCl₂.

FIG. 7 compares the stabilities of AuNP/IFNa and HA-AuNP/IFNa in NaCl 150 mM according to Experimental Example 5, wherein “1” denotes AuNP, “2” denotes AuNP/IFNα 17, “3” denotes AuNP/IFNα 120, “4” denotes HA-AuNP, “5” denotes HA-AuNP/IFNα 17, and “6” denotes HA-AuNP/IFNa 110.

FIG. 8 shows ELISA assay result of the amount of released IFNa, after reacting HA-AuNP/IFNa complex in BSA for 3 days according to Experimental Example 5.

FIG. 9 shows the results of analyzing the activities of HA-AuNP/IFNa and AuNP/IFNa complexes, PEG-Intron, and IFNa, through antiproliferation assay using daudi (burkitt lymphoma) cells.

FIG. 10 shows the stabilities of HA-AuNP/IFNa and AuNP/IFNa complexes and IFNa in human serum, through antiproliferation assay using daudi cells according to Experimental Example 7.

FIG. 11 shows cytotoxicity of HA-AuNP through MTS assay using daudi cells according to Experimental Example 8.

FIG. 12 shows the quantitative analysis result of INFa delivered to the liver of mouse by HA-AuNP/IFNa and AuNP/IFNa complexes, PEG-Intron, and IFNa, by ELISA assay, according to Experimental Example 9.

FIG. 13 shows the analysis result of distribution of HA-AuNP/IFNa according to liver cells by ICP-MS and TEM image according to Experimental Example 10.

FIG. 14 shows the analysis result of distribution of HA-AuNP/IFNa according to main tissues by ELISA and ICP-MS according to Experimental Example 11.

FIG. 15a shows the analysis result of OAS 1 by western blot, in order to examine antiviral effects of HA-AuNP/IFNa and AuNP/IFNa complexes, PEG-Intron, and IFNa, in mouse liver according to Experimental Example 12. (1:control, 2:IFNa, 3: PEG-intron, 4:HA-AuNP, 5:HA-AuNP/IFNa 120, 6:HA-AuNP/IFNa 75, 7:AuNP/IFNa 110, * P<0.05 and ** P<0.01)

FIG. 15b shows the analysis result of Mx 1 by western blot, in order to examine antiviral effects of HA-AuNP/IFNa and AuNP/IFNa complexes, PEG-Intron, and IFNa, in mouse liver according to Experimental Example 12. (1:control, 2:IFNa, 3: PEG-intron, 4:HA-AuNP, 5:HA-AuNP/IFNa 120, 6:HA-AuNP/IFNa 75, 7:AuNP/IFNa 110, * P<0.05 and ** P<0.01)

### MODE FOR INVENTION

Hereinafter, the present invention will be explained in detail with reference to Examples.

However, these Examples are only to illustrate the invention, and the present invention is not limited thereto.

### Example 1

### Preparation of HA-AuNP/IFNa Complex

<1-1> Preparation of Hyaluronic Acid (HS-HA) Derivative Having a Thiol Group Introduced at the End

200 mg of hyaluronic acid (HA) (MW 12 KDa) and 230 mg of sodium chloride (NaCl) were dissolved in 20 mL of 0.1 M borate buffer of pH 8.5, and cystamine hydrochloride was added in the amount of 1 mole per HA unit. After 2 hours, 200 mM of sodium cyanoborohydride was added and the mixture was reacted at 40° C. for 5 days. And then, 100 mM of dithiothreitol (DTT) was added, and the mixture was reacted at 25° C. for 12 days, dialyzed for 2 days for 150 mM NaCl, for 1 day for 25%(v/v) ethanol, and for 1 day for distilled water to purify, and then, freeze-dried to obtain HS-HA derivative having a thiol group introduced at the end.

Before using the HS-HA derivative, TCEP was added in the amount of 1 mole per one molecule of HA, and reacted for 12 hours to reduce disulfide bonds that may be generated during the purification process, and the TCEP was removed using PD-10 desalting column. If TCEP remains, it may influence on the structure of IFNa to be added subsequently. Through Ellman assay, it was confirmed that about 95 mole % or more of thiol groups are introduced at the end of HA (see Anal Biochem., 1973, 56(1), 310-1.).

<1-2> Preparation of HA-AuNP

10 mg of chloroauric acid was dissolved in 90 mL of distilled water and the solution was heated until boiling. When the solution began to boil, 5 mL of 25 mM sodium citrate was added, and the mixture was reacted for about 30 minutes until it turned to dark red, to obtain a gold nanoparticle solution.

To 50 mL of the prepared gold nanoparticle solution (5.4 nM), 820 μg of the HS-HA obtained in the <1-1> was added, and the mixture was reacted for 1 day to obtain HA-modified gold nanoparticles (HA-AuNP).

<1-3> Preparation of HA-AuNP/IFNa Complex

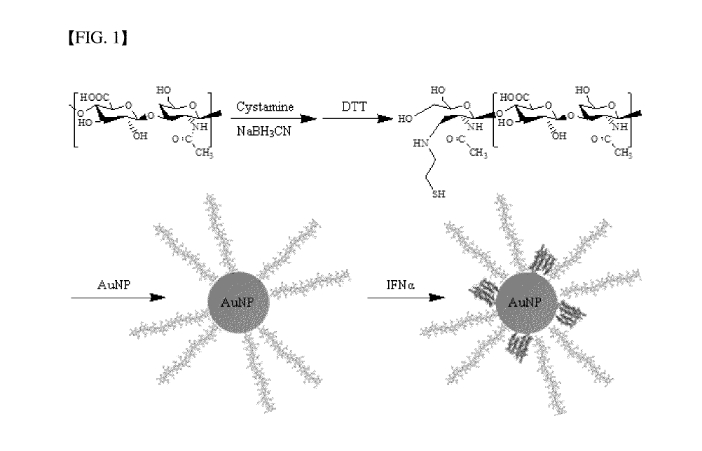

Next, interferon alpha (IFNa) (Shin Poong Pharm. Co. Ltd.) was dissolved in PBS (pH 7.4) at a concentration of 0.7 mg/mL, and the solution was introduced into 10 mL of the 5 to 6 nM HA-AuNP solution obtained in the <1-2> so that the number of interferon may be 10 to 200 per gold nanoparticle, and then, the mixture was reacted for 12 hours. And then, gold nanoparticles were precipitated using centrifuge (20,000×g, 20 minutes) and supernatant was filtered, which was repeated twice to remove unreacted interferon, and finally, the residue was redispersed in PBS (pH 7.4) to obtain HA-AuNP/IFNa complex (FIG. 1).

### Comparative Example 1

### Preparation of AuNP/IFNa Complex

10 mg of chloroauric acid was dissolved in 90 mL of distilled water and the solution was heated until boiling. When the solution began to boil, 5 mL of 25 mM sodium citrate was added, and the mixture was reacted for about 30 minutes until it turned to dark red, to obtain a gold nanoparticle (AuNP) solution.

Next, interferon alpha (IFNa) (Shin Poong Pharm. Co. Ltd.) was dissolved in PBS (pH 7.4) at a concentration of 0.7 mg/mL, and the solution was introduced into the gold nanoparticle solution so that the number of interferon may be 10 to 200 per gold nanoparticle, and then, the mixture was reacted for 12 hours. And then, gold nanoparticles were precipitated using centrifuge (20,000×g, 20 minutes) and supernatant was filtered, which was repeated twice to remove unreacted interferon, and finally, the residue was redispersed in PBS (pH 7.4) to obtain AuNP/IFNa complex wherein IFNa is bound to non-surface modified AuNP.

### Experimental Example 1

### Confirmation of Formation of HA-AuNP/IFNa Complex and Analysis

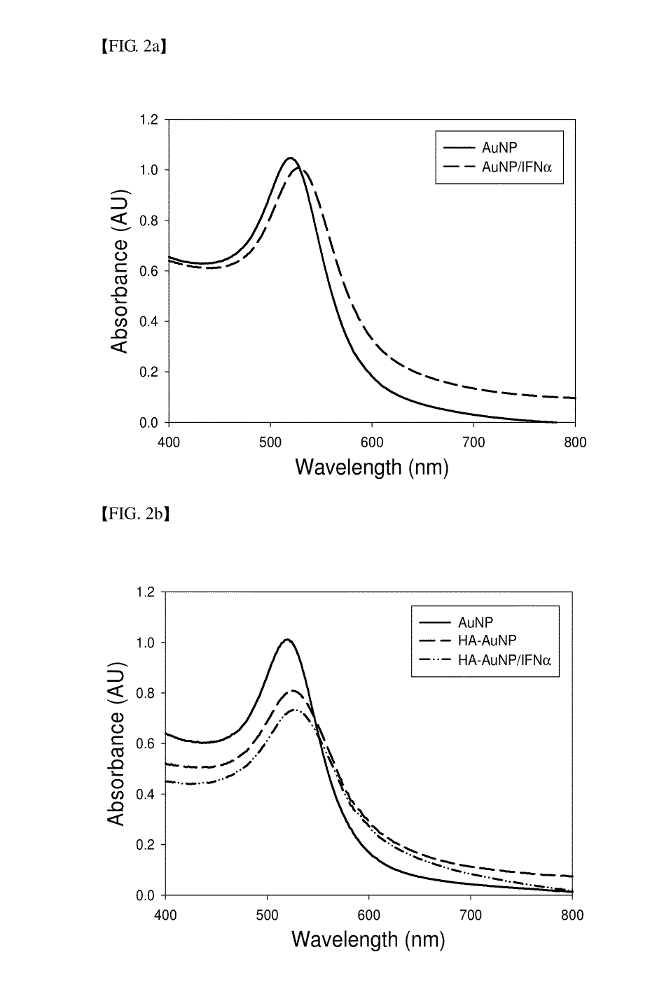

The AuNP/IFNa complex prepared according to <Comparative Example 1> wherein IFNa is bound to non-surface modified AuNP, and the HA-AuNP/IFNa complex prepared according to <Example 1> wherein IFNa is bound to AuNP that is surface modified by HA (each complex was prepared by introducing 200 interferon per gold nanoparticle) were comparatively analyzed using DLS (dynamic light scattering) (Zetasizer Nano, Malvern Instrument Co., UK) and UV-Vis spectra (S-3100, Scinco Co., Seoul, Korea). As the result of analysis, it was confirmed that in the case of HA-AuNP/IFNa, about 110 IFNa was bound, and in the case of AuNP/IFNa, about 120 IFNa was bound.

Wherein, for comparison, all the solution was dispersed in DIW, and then, analyzed. As shown in FIG. 2a, when IFNa was added to AuNP, red shift of surface plasmon resonance peak was confirmed. And, as shown in FIG. 2b, when HA was introduced into AuNP and IFNa was added, red shift of surface plasmon resonance peak was also confirmed.

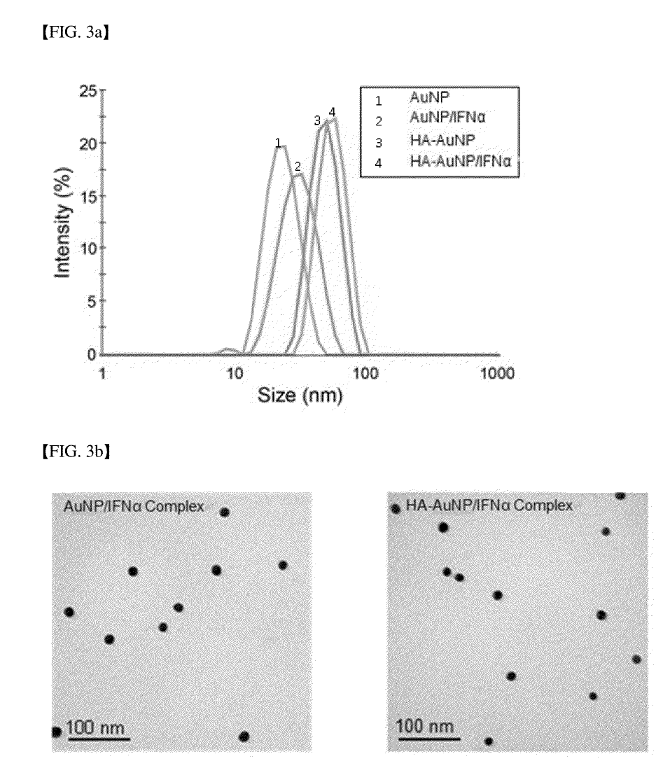

As the result of measuring hydrodynamic diameter using DLS, it was confirmed that the diameter of AuNP was 22.16 nm, and the diameter increased to 29.25 nm after adding IFNa. Thus, it can be seen that IFNa was bound to AuNP in a single layer (FIG. 3a). Meanwhile, it was confirmed that the diameter after introducing HA into AuNP was 52.23 nm, and the diameter increased to 57.83 nm after adding IFNa (FIG. 3a). It was also confirmed that the complex was mono dispersed through TEM image (FIG. 3b).

As such, the measurement results using DLS, UV-Vis spectra showed that IFNa was bound to AuNP even after introducing HA into AuNP.

### Experimental Example 2

### Quantitative Analysis of HA-AuNP/IFNa Complex

The number of IFNa bound to HA-AuNP was quantitatively analyzed through ELISA assay. Specifically, the complex was formed so that the molecular number of IFNa may be 10 to 200 per AuNP in HA-AuNP (5.4 nM) according to Example 1, and then, centrifuged (20,000×g, 20 minutes) to precipitate, and supernatant part and AuNP-bound part were separately diluted with PBS, and then, quantitative analysis was conducted using IFNa as a standard curve (obtained by diluting IFNa respectively to 0, 0.015625 0.3125, 0.625, 1.25, 2.5, 5, 10 ng/mL with PBS, and conducting ELISA assay), using ELISA assay kit (VeriKine™ Human Interferon-Alpha ELISA Kit, PBL InterferonSource, Piscataway, N.J.), according to manufacturer's instruction. As the experiment result, detection degree of IFNa bound to AuNP by ELISA assay was different according to the number of bound molecules. This is considered to be due to steric hindrance, or because the activity of IFNa may be decreased when the antibody-binding part of IFNa binds to gold nanoparticles. Thus, IFNa in the supernatant, which was not bound to gold nanoparticles was quantified.

As explained, if the structure of IFNa changes or the activity decreases due to interaction with AuNP, IFNa in the supernatant may not be detected by ELISA assay, and thus, for more exact decision, the molecular number of IFNa binding to HA-AuNP was analyzed with fluorescence spectrofluorophotometer (Fluoroskan Ascent FL, Lab systems, Germany). Wherein, only one FITC was bound per one IFNa molecule to minimize disturbance when binding to AuNP surface. Specifically, 1 mg of IFNa was added to 1 ml of 0.1M sodium carbonate buffer, and then, fluorescein isothiocyanate (FITC) was introduced in the amount of 3 to 5 moles per IFNa, and the mixture was reacted at room temperature for 1 hour, and then, dialyzed in PBS (pH 7.4) for 24 hours to remove non-bound IFNa. As explained above, a complex was formed so that the number of IFNa-FITC may be 10 to 200 per AuNP, and then, it was precipitated using centrifuge (20,000×g, 20 minutes), and the supernatant was analyzed through fluorescence analyzer, and IFNa bound to HA-AuNP was quantitatively analyzed with IFNa-FITC as a standard curve (IFNa-FITC was made to 1, 2, 4, 8, 16, 32 μg/mL, based on IFNa, and simultaneously analyzed by fluorescence analyzer). For AuNP without HA, experiment was conducted by the same method.

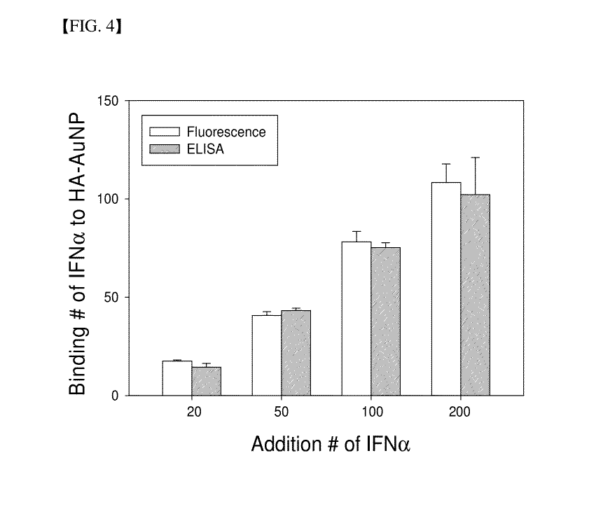

As the results, it can be seen that the number of bound IFNa increases according to the added amount of IFNa, when 20, 50, 100 and 200 IFNa were introduced into HA-AuNP per AuNP, and that about 17, 110 IFNa were bound respectively when 20 and 200 IFNa were introduced, showing that ELISA assay results are almost identical to fluorescence analysis results (FIG. 4). Meanwhile, it can be seen that the maximum molecular number of IFNa binding to AuNP without HA was about 120 when 200 IFNa was added per AuNP, thus showing that more IFNa are bound compared to HA-introduced AuNP (results not shown).

Thus, it was confirmed that IFNa can be bound to the surface of non-surface modified AuNP, thus showing that the maximum amount of protein binding to metal nanoparticles may be controlled according to introduction of HA and the used amount.

### Experimental Example 3

### Analysis of IFNa Binding Mechanism to AuNP

To analyze IFNa binding mechanism to AuNP in the HA-AuNP/IFNa prepared in <Example 1> (the case wherein 200 IFNa is added per AuNP and 120 IFNa is bound, and the case wherein 200 IFNa is added per HA-AuNP and 110 is bound), a chemical agent disturbing a specific interaction was added to remove the bonding. Specifically, the HA-AuNP/IFNa complex (20 μg/mL based on IFNa, 1 mL) was treated with Tween 20 (for confirmation of hydrophobic interaction) at a concentration of 1%(v/v), or treated MgCl₂ (for confirmation of electrostatic attraction) in an amount of 1M. And, it was treated with Tween 20 and MgCl₂ respectively at 1%(v/v), 1 M. After 6 hours, AuNP was precipitated using centrifuge (20,000×g, 20 minutes), and the supernatant was analyzed through ELISA assay to quantitatively analyze IFNa released from AuNP, which is shown in the following Table 1.

TABLE 1

| Reagent | MgCl₂ | Tween 20 | MgCl₂ + Tween 20 |
|---|---|---|---|
| The ratio of IFNa | 10.07% | 40.62% | 95.03% |
| released from | | | |
| HA-AuNP/IFNa(%) | | | |

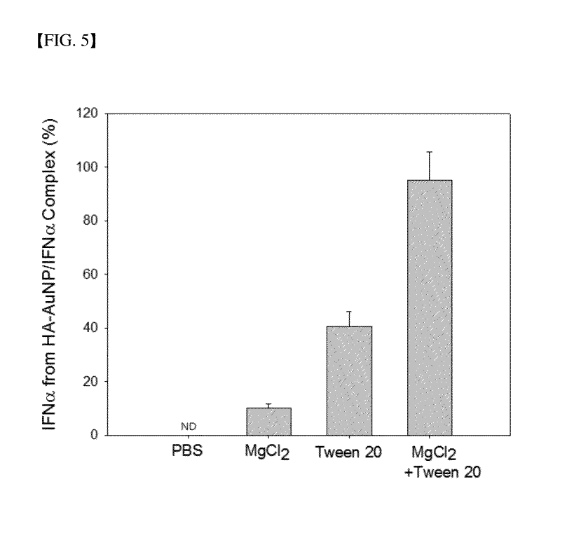

As shown in the Table 1, about 10.07% of IFNa was released by Tween 20, and about 40.62% of IFNa was released by MgCl₂. Thus, it was confirmed that IFNa is bound to AuNP mainly by hydrophobic interaction. It was also confirmed that about 95.03% was released when treated with Tween 20 and MgCl₂ together. Thus, it can be seen that IFNa is bound to the surface of AuNP through both hydrophobic interaction and electrostatic attraction (Table 1). AuNP/IFNa complex shows similar aspect (FIG. 5).

### Experimental Example 4

### Confirmation of Structure Change of IFNa when Forming HA-AuNP/IFNa Complex

1) Analysis Method

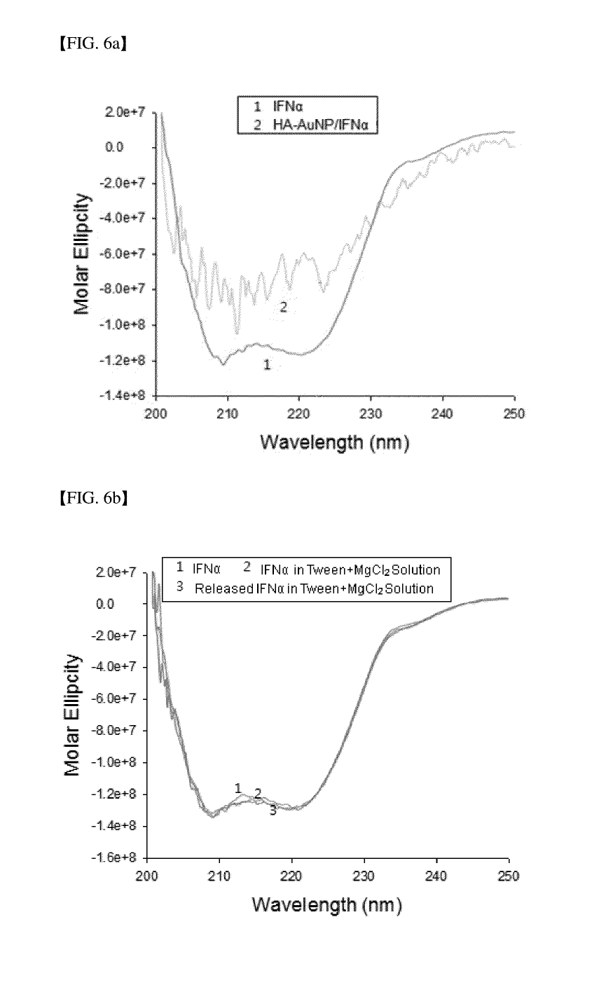

Circular Dichroism analysis of IFNa was conducted for the HA-AuNP/IFNa complex prepared according to <Example 1> and for the case wherein the chemical agents (Tween 20 and MgCl₂) were added according to Experimental Example 3, based on the concentration of IFNa (0.1 mg/ml), and the results are shown in FIG. 6a and FIG. 6b. Analysis conditions are as follows.

<CD Analysis Conditions>

UV spectrophotometer: JASCO J-715

Measurement conditions: 25° C., 200˜250 nm, N2 atmosphere

A quartz cuvette: 2 mm path length

Raw data: 0.2 mm interval according to 1 second reaction time

2) Analysis Results

As shown in FIG. 6a, in the case of HA-AuNP/IFNa, CD value cannot be properly obtained due to scattering by gold nanoparticles and energy transfer of gold nanoparticles from protein.

Thus, HA-AuNP/IFNa (0.1 mg/mL based on IFNa, 500 μl) was treated with Tween 20 and MgCl₂ respectively at 1%(v/v) and 1 M, and gold nanoparticles were precipitated with centrifuge (20,000×g, 20 minutes) after 6 hours, and then, IFNa in the supernatant was quantified by ELISA and analyzed by CD. As shown in FIG. 6b, CD peak did not change even if IFNa was treated with tween 1% and MgCl₂ 1M, and it was confirmed that the CD peak correspond to the CD peak of IFNa released from gold nanoparticles. Thus, it was confirmed that 3D structure of protein (IFNa) does not change when IFNa binds to gold nanoparticles.

### Experimental Example 5

### Stability Evaluation of HA-AuNP/IFNa Complex in Plasma

1) Stability Evaluation in NaCl 150 mM

To evaluate plasma stability of drug delivery system wherein protein is bound to the surface of surface modified metal nanoparticles, stabilities of AuNP, AuNP/IFNa complex prepared according to <Comparative Example 1> wherein IFNa is bound to non-surface modified AuNP, and HA-AuNP/IFNa complex prepared according to <Example 1> wherein IFNa is bound to AuNP that is surface modified by HA were evaluated in NaCl 150 mM.

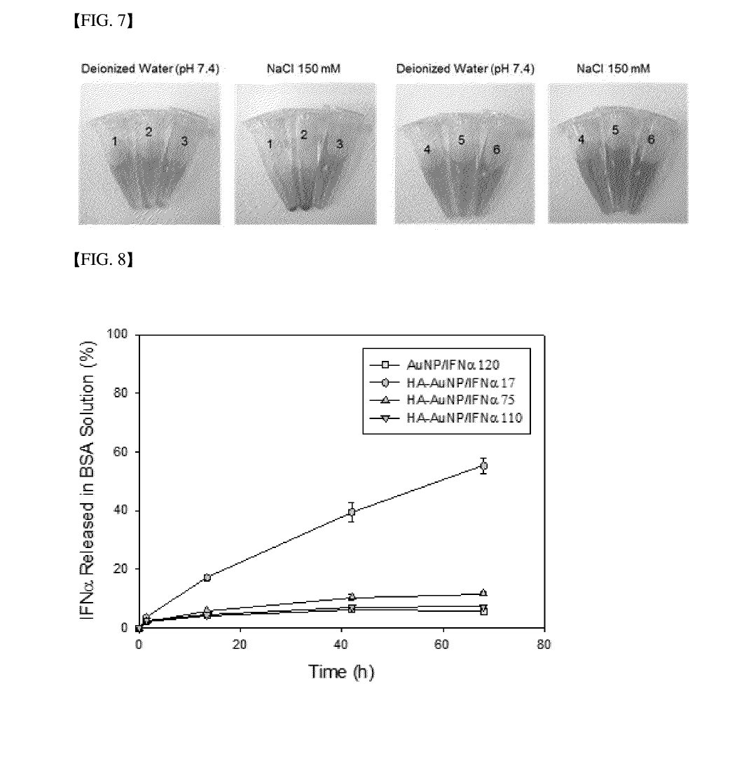

As shown in FIG. 7, in the case of AuNP and the AuNP/IFNa complex without HA of <Comparative Example 1>, a complex wherein IFNa was not completely bound (for example: AuNP/IFNa 17) showed precipitation within a short time in NaCl 150 mM, but AuNP/IFNa 120 complex wherein IFNa is maximally bound was comparatively stable in NaCl 150 mM. However, since HA-AuNP itself is very stable in NaCl 150 mM, all the HA-AuNP/IFNa complexes were very stable in NaCl 150 mM regardless of the number of IFNa.

2) Stability Evaluation in Plasma Protein

Next, stability in plasma protein was evaluated. IFNa bound to HA-AuNP/IFNa complex may be released due to competitive interaction of plasma protein with AuNP in the body. Particularly, plasma albumin is known to bind AuNP well. Thus, to test stability of HA-AuNP/IFNa complex in plasma, the amount of IFNa released by bovine serum albumin (BSA) was quantitatively analyzed.

Specifically, HA-AuNP/IFNa complexes were formed according to <Example 1> so that the number of IFNa per AuNP may become 17, 43, 75 and 110, respectively (prepared by adding 20, 50, 100, 200 IFNa per AuNP). A BAS solution (Sigma-Aldrich, St. Louis, Mo.) was added to each complex at a concentration of 2 mg/mL, and then, the mixture was reacted for 3 days while gently shaking. After 3 days, it was precipitated using centrifuge (20,000×g, 20 minutes), supernatant was analyzed through ELISA assay by the same method as Experimental Example 2, and IFNa released from HA-AuNP was quantitatively analyzed with IFNa as a standard curve.

As the result, as shown in FIG. 8, it was confirmed that in the case of HA-AuNP/IFNa 17 complex, when reacted with BSA for 3 days, IFNa was rapidly released. However, as the molecular number of IFNa per AuNP increases, IFNa released by BSA decreased, and in the case of HA-AuNP/IFNa 75, HA-AuNP/IFNa 110, AuNP/IFNa 120 complexes, IFNa was not substantially released. This is considered to be because the opportunity of interaction of AuNP and BSA decreases as the molecular number of IFNa per AuNP increases.

### Experimental Example 6

### In Vitro Analysis of the Activity of HA-AuNP/IFNa Complex

Daudi cell, which is human B-lymphoblastoid cell, is known to be a cell that does not proliferate well when interferon exists, and thus, is used a lot for test of the activity of interferon.

Thus, Daudi cell (Korean Cell Line Bank) was dispersed in cell culture medium (RPMI 1640 media supplemented with 10 vol % fetal bovine serum (FBS) and 10 IU/mL of antibiotics (penicillin), GIBCO) at 4×10⁵ cells/mL, and then, each 50 μL was introduced into 96 well plate. And, IFNa, PEG-Intron (Shin Poong Pharm. Co. Ltd.), HA-AuNP/IFNa 75, HA-AuNP/IFNa 110 and AuNP/IFNa 120 complexes were diluted to various concentrations using cell culture medium, and each 50 μL was introduced into daudi cells. It was cultured under conditions of 37° C. and 5% CO2 for 4 days, and then, proliferation rate of daudi cells was confirmed through MTS assay (Cell Titer 96 AQueous One Solution Reagent, Promega (Madison, Wis.)).

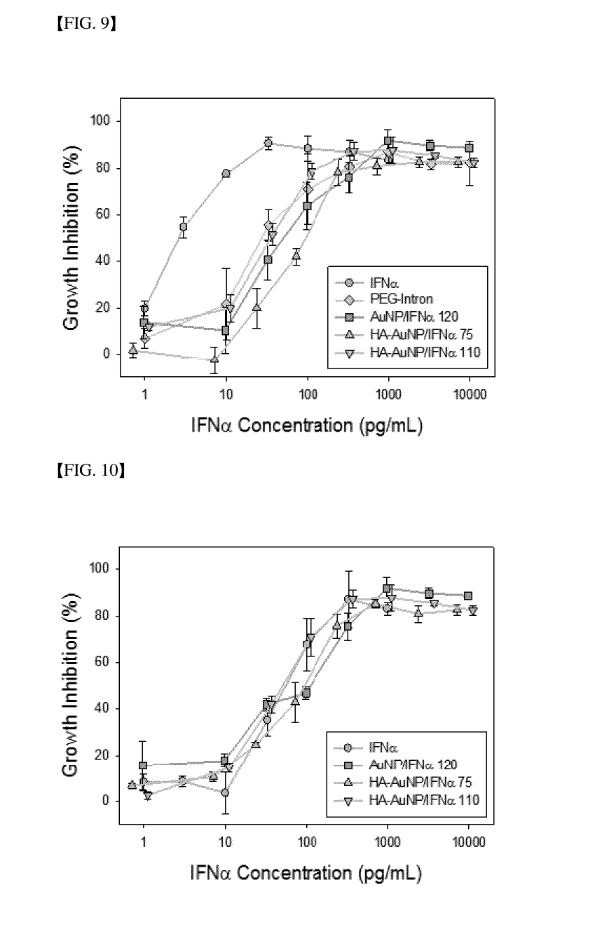

As shown in FIG. 9, although the activities of HA-AuNP/IFNa and AuNP/IFNa complexes are low compared to IFNa, they show equal efficacy to commercially available PEG-Intron. However, even if in vitro activity of IFNa to cells is similar, it has been reported that particles with a size of 10 nm˜20 nm may reduce renal clearance in vivo (Annu. Rev. Biomed. Eng. 2011, 13, 507-530.), and since the complex according to the Examples of the present invention has liver targeted effect by HA, higher efficiency and longer duration than commercially available PEG-Intron may be expected in vivo.

### Experimental Example 7

### In Vitro Analysis of Activity of HA-AuNP/IFNa Complex in Human Serum

IFNα, PEG-Intron, AuNP/IFNα 120, HA-AuNP/IFNα 75, HA-AuNP/IFNα 110 complexes were respectively dissolved in human serum (Sigma-Aldrich, St. Louis, Mo.) so that the concentration of IFNa may be identically 20 μg/mL, and reacted at 37° C. for 3 days. After 3 days, biological activity of each sample was measured by MTS assay using daudi cells by the same method as Experimental Example 6.

As shown in FIG. 10, it was confirmed that IFNa was rapidly decomposed in human serum and the activity decrease to 1/10. To the contrary, the activities of HA-AuNP/IFNa 75, HA-AuNP/IFNa 110 and AuNP/IFNa 120 complexes did not decrease, thus confirming that the drug delivery system according to Examples of the present invention has very high serum stability, and it can be seen that the stability further increases as the molecular number of bound protein per metal nanoparticle increases.

### Experimental Example 8

### Confirmation of HA-AuNP Cytotoxicity

To confirm cytotoxicity of interferon alpha delivery system HA-AuNP, experiment was conducted in daudi cells by the same method as <Experimental Example 6> and the result is shown in FIG. 10.

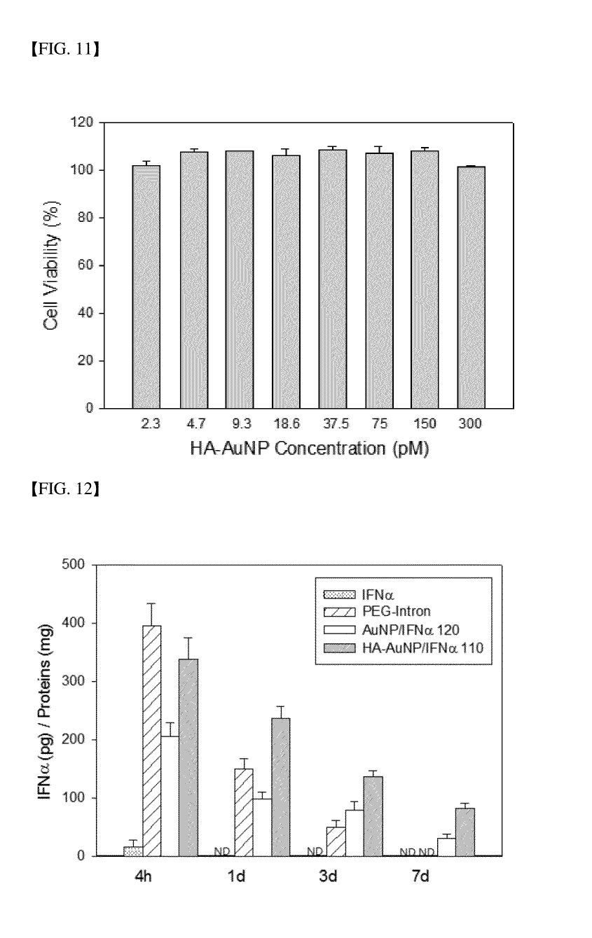

As shown in FIG. 11, it was confirmed that there is no distinct cytotoxicity even at 10 times higher concentration than the maximum concentration of HA-AuNP used in <Experimental Example 8>.

### Experimental Example 9

### Evaluation of Liver Targeted Delivery of HA-AuNP/IFNa Complex (In Vivo)

PBS, IFNa, PEG-Intron, HA-AuNP, HA-AuNP/IFNa 75, HA-AuNP/IFNa 110, AuNP/IFNa 120 complexes were respectively introduced through tail vein of Balb/c mouse (POSTECH Biotechnology center, female Balb/c mice, 5 week-aged, about 20 g) (injection amount: 0.2 mg/kg based on interferon), and then, after 4 hours, 1 day, 3 days and 7 days, liver was respectively collected, and the concentration of IFNa (pg) to total protein (mg) extracted from the liver was quantified by ELISA assay and shown in FIG. 12.

As shown in FIG. 12, HA-AuNP itself did not influence on the IFNa concentration in the liver. IFNa was not detected at 1 day, 3 day and 7 day, and PEG-Intron was detected at 3 day at a concentration of about 100 pg/mg, but not detected at 7 day. This is considered to be because IFNa and PEG-Intron are rapidly removed in the body. To the contrary, AuNP-based IFNa complexes according to the Examples of the present invention showed higher IFNa level than PEG-Intron at 3 day, and although IFNa physically binds to AuNP instead of covalent bonding, it still remained in the liver tissue at 7 day. Particularly, it was confirmed that HA-AuNP/IFNa 110 complex exist more in the liver tissue.

This is considered to be because nanoparticles having a size of 10 nm or more may function for preventing rapid removal by renal filtration or urinary excretion (nnu Rev Biomed Eng. 2011; 13:507-30), antifouling property of HA may reduce intake of nanoparticles by reticuloendothelial system (RES) or circulating macrophages) and prevent enzymatic decomposition of IFNa, and HA may function for liver targeted delivery (Nano Lett 2011; 11:2096-103 and Adv Mater 2008; 20(21):4154-7).

### Experimental Example 10

### Liver Cell Distribution of HA-AuNP/IFNa Complex (In Vitro)

PBS, AuNP/IFNα 120, HA-AuNP/IFNα 110 complexes were respectively introduced through tail vein of Balb/c mouse (injection amount: 0.2 mg/kg, based on interferon), and after 1 day, liver of the mouse was collected and the distribution of AuNP according to liver cells was analyzed by ICP-MS and TEM image.

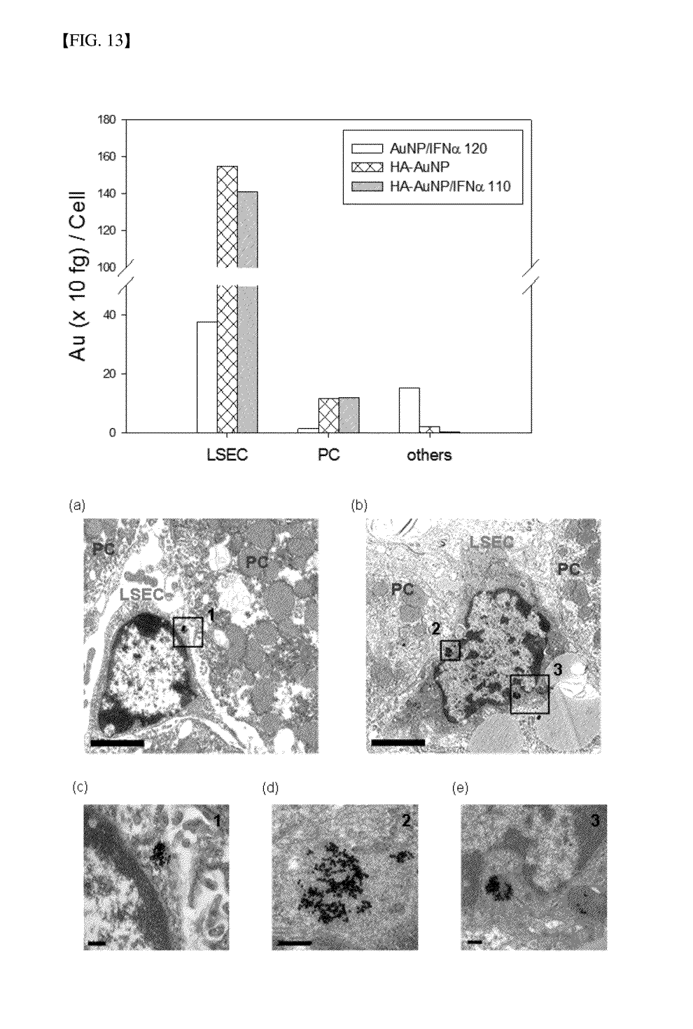

As shown in FIG. 13, it was confirmed that HA-AuNP/IFNα120 complex is mainly distributed in liver sinusoidal endothelial cell (LSEC).

### Experimental Example 11

### Distribution of HA-AuNP/IFNα Complex According to Main Tissues (In Vivo)

PBS, AuNP/IFNa 120, HA-AuNP/IFNa 110 complexes were respectively introduced through tail vein of Balb/c mouse (injection amount: 0.2 mg/kg, based on inteferon), and after 1 day, liver, spleen, kidney and lung of the mouse were collected, and the amount of IFNα and the amount of AuNP in the liver were respectively quantified by ELISA and ICP-MS.

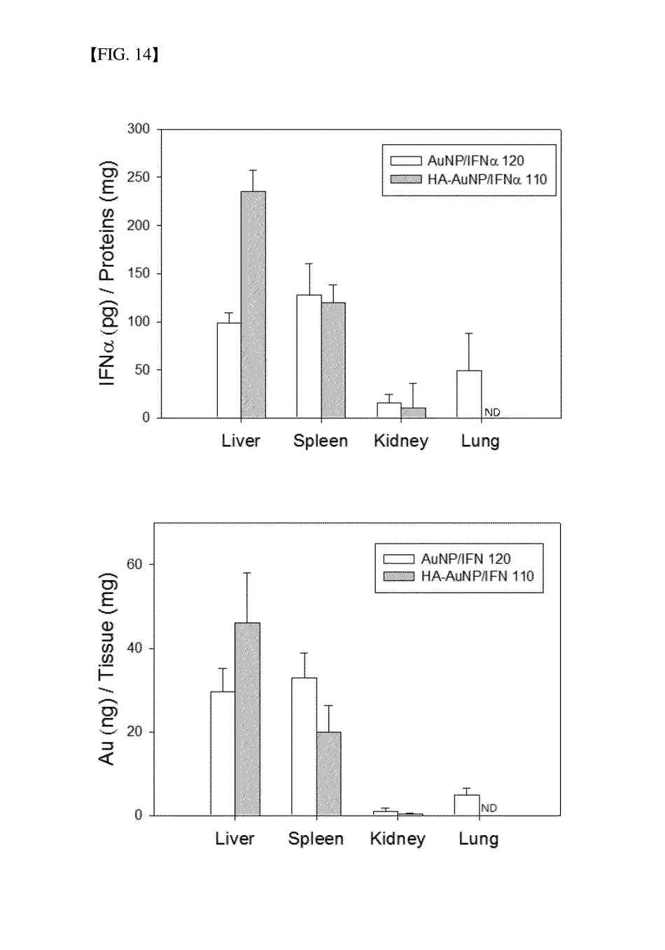

As shown in FIG. 14, HA-AuNP/IFNα120 had higher amounts of IFNα and AuNP in the liver, but lower amounts of IFNα and AuNP in the lung than AuNP/IFNα110 complex. It shows that HA aids in liver targeted delivery of the complex.

### Experimental Example 12

### Analysis of Liver Targeted Delivery Efficiency of HA-AuNP/IFNa Complex (In Vivo)

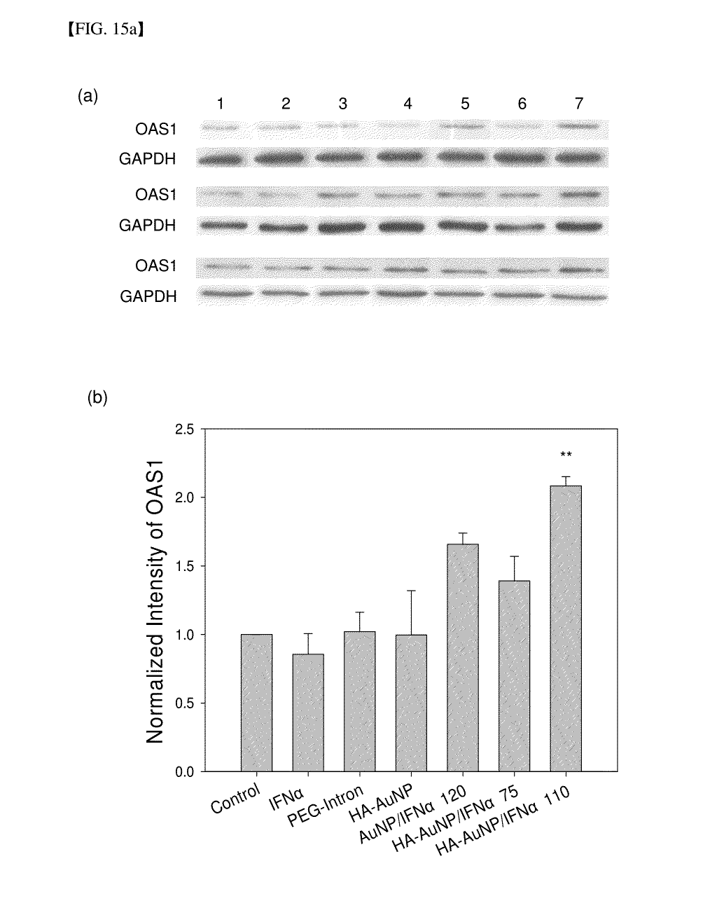

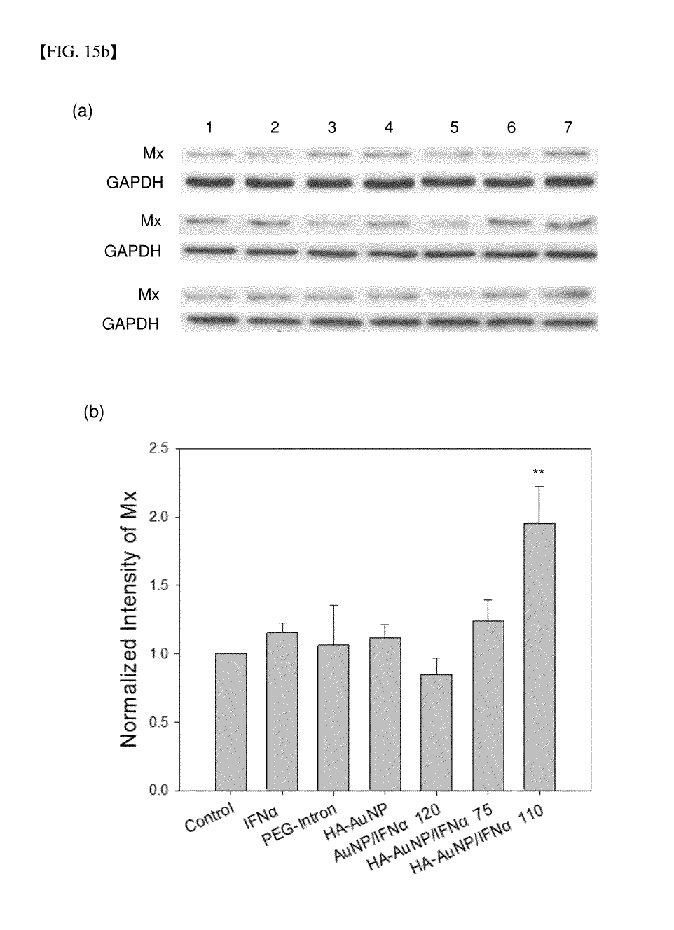

PBS, IFNa, PEG-Intron, HA-AuNP, HA-AuNP/IFNa 75, HA-AuNP/IFNa 110, AuNP/IFNa 120 complexes were respectively introduced through tail vein of Balb/c mouse (POSTECH Biotechnology center, female Balb/c mice, 5 week-aged, about 20 g) (injection amount: 0.2 mg/kg based on interferon), and after 7 days, liver of the mouse was collected, and the levels of 2′-5′-oligoadenylate synthetase 1 (OAS 1) and myxovirus resistance (Mx) in the liver were quantified through western blot and shown in FIG. 15a (OAS 1) and FIG. 15b (Mx) (Biomaterials, 2011, 32, 8722-8729).

The OAS 1 and Mx are proteins expressed by interferon and have antiviral property. As shown in FIG. 15a, it was confirmed that in the case of HA-AuNP/IFNa 75, HA-AuNP/IFNa 110 and AuNP/IFNa 120 complexes, the level of OAS1 which functions for antivirus in the liver remarkably increases even after 7 days compared to IFNa and PEG-intron, and particularly, it was confirmed that HA-AuNP/IFNa 110 complex expresses higher level of OAS 1 than HA-AuNP/IFNa 75 and AuNP/IFNa 120 complexes.

And, as shown in FIG. 15b, it was confirmed that HA-AuNP/IFNa 110 complex expresses higher level of Mx compared to IFNa, PEG-intron, HA-AuNP/IFNa 75, and AuNP/IFNa 120 complex. This corresponds to the result of IFNa concentration in the liver tissue as confirmed in <Experimental Example 9>.

1. Liver targeted drug delivery system comprising metal nanoparticles surface modified by dextran, heparin, hyaluronic acid, a salt thereof, or a derivative thereof, and peptide or protein drug bound to the non-modified surface of the metal nanoparticles.

2. The liver targeted drug delivery system according to claim 1, wherein the dextran, heparin, hyaluronic acid, a salt thereof, or a derivative thereof has molecular weight of 5,000 to 20,000 Da.

3. The liver targeted drug delivery system according to claim 1, wherein the metal nanoparticle is gold nanoparticle, silver nanoparticle, or magnetic nanoparticle.

4. The liver targeted drug delivery system according to claim 1, wherein the dextran, heparin, hyaluronic acid, a salt thereof, or a derivative thereof is bound in a molecular number of 10 to 100 per one metal nanoparticle so that the metal nanoparticles are surface modified.

5. The liver targeted drug delivery system according to claim 4, wherein hyaluronic acid derivative of the following Chemical Formula 1 is bound to the surface of the metal nanoparticle through an end functional group (R in the Chemical Formula 1) so that the metal nanoparticles are surface modified. 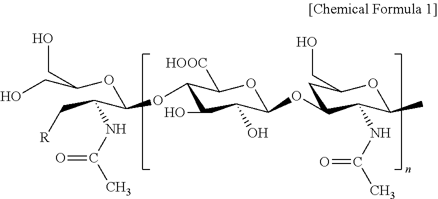
in the Chemical Formula 1,
n is an integer of from 12 to 50, R is NH(CH₂)mR², m is an integer of from 2 to 10, and R² is C₆H₁₂O₂ or SH.

6. The liver targeted drug delivery system according to claim 1, wherein the peptide or protein drug is bonded in a molecular number of 10 to 200 per one metal nanoparticle.

7. The liver targeted drug delivery system according to claim 1, wherein the peptide or protein drug is covalently or non-covalently bonded to the surface of the metal nanoparticles.

8. The liver targeted drug delivery system according to claim 7, wherein the peptide or protein drug is covalently bonded to the surface of the metal nanoparticles, and cysteine that does not form a disulfide bond is included in the amino acid constituting the drug.

9. The liver targeted drug delivery system according to claim 7, wherein the peptide or protein drug is non-covalently bonded to the surface of the metal nanoparticles, and at least one amino acid selected from the group consisting of tyrosine, lysine, aspartice acid, arginine, hystidine and tryptophan is included in the amino acid constituting the drug.

10. The liver targeted drug delivery system according to claim 1, wherein the peptide or protein drug is selected from the group consisting of TNF-related apoptosis-inducing ligand, vascular adhesion protein 1, hepatocyte growth factor and interferon alpha (IFNa).

11. A method for preparing liver targeted drug delivery system comprising
(1) introducing material having an end amine group and an internal disulfide bond or catecholamine-based material in dextran, heparin, hyaluronic acid, a salt thereof, or a derivative thereof;
(2) reacting the introduced material with the surface of metal nanoparticles to prepare surface-modified metal nanoparticles; and
(3) binding peptide or protein drug to the non-modified surface of the metal nanoparticles.

12. The method according to claim 11, wherein the step (1) comprises
(1-1) introducing material having an end amine group and an internal disulfide bond in dextran, heparin, hyaluronic acid, a salt thereof, or a derivative thereof; and
(1-2) cutting the disulfide bond formed through the step (1-1) using at least one reducing agent selected from the group consisting of dithiothreitol (DTT), 2-mercaptoethanol, and tris(2-carboxyethyl) phosphine, (TCEP).

13. The method according to claim 11, wherein the step (1) comprises introducing material having an amine end group and an internal disulfide bond or catecholamine-based material in the hyaluronic acid, a salt thereof, or a derivative thereof to prepare hyaluronic acid derivative of the following Chemical Formula 1: 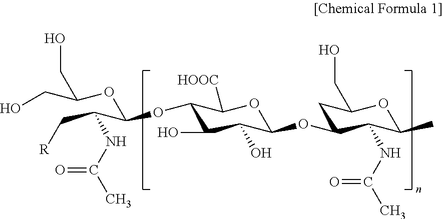
in the Chemical Formula 1,
n is an integer of from 12 to 50, R is NH(CH₂)mR², m is an integer of from 2 to 10, and R² is C₆H₁₂O₂ or SH.

14. The method according to claim 11, wherein the dextran, heparin, hyaluronic acid, a salt thereof, or a derivative thereof has molecular weight of 5,000 to 20,000 Da.

15. The method according to claim 11, wherein in the step (2), the dextran, heparin, hyaluronic acid, a salt thereof, or a derivative thereof is reacted in a molecular number of 10 to 100 per one metal nanoparticle

16. The method according to claim 11, wherein the material having an end amine group and an internal disulfide bond is selected from the group consisting of 2,2′-disulfanediyldiethanamine, 3,3′-disulfanediyldipropan-1-amine, 4,4′-disulfanediyldibutan-1-amine, 5,5′-disulfanediyldipentan-1-amine, and a salt thereof.

17. The method according to claim 11, wherein the catecholamine-based material is selected from the group consisting of dopamine, norepinephrine, and a salt thereof.

18. The method according to claim 11, wherein in the step (2), the metal nanoparticle is gold nanoparticle, silver nanoparticle or magnetic nanoparticle.

19. The method according to claim 11, wherein in the step (3), peptide or protein drug is covalently bonded to the non-modified surface of the metal nanoparticles, and cysteine that does not form a disulfide bond is included in the amino acid constituting the peptide or protein drug.

20. The method according to claim 11, wherein in the step (3), peptide or protein drug is non-covalently bonded to the non-modified surface of the metal nanoparticles, and at least amino acid selected from the group consisting of one tyrosine, lysine, aspartice acid, arginine, hystidine and tryptophan is included in the amino acid constituting the peptide or protein drug.

21. The method according to claim 11, wherein in the step (3), IFNa is electrostatically and hydrophobically bonded to the non-modified surface of the metal nanoparticles.

22. A pharmaceutical composition for preventing or treating liver disease comprising the liver targeted drug delivery system according to claim 1.

23. The pharmaceutical composition according to claim 22, wherein the disease is acute hepatitis, chronic hepatitis, liver cirrhosis, cirrhosis, fatty liver, or liver cancer.
